(19) United States
(12) Patent Application Publication
(10) Pub. No.: US 2021/0254113 A1
(43) Pub. Date: Aug. 19, 2021

(54) Production of Bacterial Polysaccharides

(71) Applicant: MSD Wellcome Trust Hilleman Laboratoriees PVT., Ltd., New Dehilhi (IN)

(72) Inventors: Sandeep Sharma, Uttar Pradesh (IN); Nitin Kumar, Uttar Pradesh (IN); Sarmad Hanif, New Delhi (IN); Manoj Kuma Chhikara, Delhi (IN); Davinder Gill, New Dehilhi (IN)

(21) Appl. No.: 16/628,405

(22) PCT Filed: Apr. 26, 2018
(86) PCT No.: PCT/IN2018/050255

§ 371 Date: Jan. 3, 2020

(30) Foreign Application Priority Data
May 17, 2017 (IN) 20171107277

(51) Int. Cl. C12P 19/04; C12N 1/20; A61K 39/095

(52) U.S. Cl. CPC C12P 19/04; A61K 39/095; C12N 1/20

(57) ABSTRACT

The present invention particularly relates to culture media composition, feed composition, and fermentation conditions for production of Neisseria meningitidis polysaccharides. The present invention describes a rapid, industrially scalable, cost effective process for the production of Neisseria meningitidis. The N. meningitidis polysaccharides of the present invention are capable of being used in the production of economical polysaccharide protein conjugate vaccine(s) against meningococcal infections.

### RELATED APPLICATIONS

This application U.S. National stage entry of International Application No. PCT/IN2018/050255, which designated the United States and was filed on Apr. 26, 2018, published in English which claims priority to Indian Application No. 201711017277, filed May 17, 2017. The entire teachings of the above applications are incorporated herein by reference.

### FIELD OF THE INVENTION

The present invention relates to an improved process of production of bacterial polysaccharides. The present invention particularly relates to culture media composition, feed composition, fermentation conditions and purification process for production of Neisseria meningitidis polysaccharides. The N. meningitidis polysaccharides of the present invention are capable of being used in the production of economical polysaccharide protein conjugate vaccine(s) against meningococcal infections.

### BACKGROUND OF THE INVENTION

Neisseria meningitidis, often referred to as meningococcus, is a Gram-negative bacterium that can cause meningitis and other forms of meningococcal disease such as meningococcemia.

On the basis of the type of capsular polysaccharide present on N. meningitidis (Men), thirteen serogroups have been identified and among the 13 identified capsular types of N. meningitidis, six (A, B, C, W135, X, and Y) account for most meningococcal disease cases worldwide. MenA has been the most prevalent serogroup in Africa and Asia but is rare/practically absent in North America. In Europe and United States, serogroup B (MenB) is the predominant cause of disease and mortality, followed by serogroup MenC and MenW. In recent past, MenX outbreaks have started showing up in sub-Saharan Africa. The multiple serogroups have hindered development of a universal vaccine for meningococcal disease.

The production of the first meningitis polysaccharide vaccine was accomplished in 1978 as there was an urgent need to combat this fatal disease. Later it was observed that the plain polysaccharide based vaccines were not very efficient in children below two years of age. These observations led to further research which revealed that infants have an immature immune system and cannot elicit immune response against plain polysaccharides.

The immune response may be characterised as T-cell dependent (TD) immune response and T-cell independent (TI) immune response. Proteins and peptides are known to elicit TD antigens by stimulating the helper T lymphocytes and generating memory cells. In contrast, polysaccharides belong to the TI antigens which do not induce T-cell activation and do not form any memory B cells, which is a major drawback while dealing with infants as they have an immature immune system.

Thus, there was a need for conjugating the bacterial polysaccharide to a protein carrier which induces a T-cell-dependent immune response characterized by increased immunogenicity among infants, prolonged duration of protection and in the reduction of nasopharyngeal carriage of meningococci. This need was fulfilled by ingenious research resulting in the production of polysaccharide-protein conjugate vaccines and the first meningococcal conjugate vaccine was licensed in United Kingdom in 1999.

The polysaccharides, especially antigenic polysaccharides, used in preparation of vaccines may be monovalent, bivalent and poly (multi) valent vaccines containing one, two or more polysaccharides, respectively. These are readily available in the market for prevention of certain diseases or infections caused by various microorganisms. The multivalent polysaccharide based vaccines have been used for many years and have proved valuable in preventing diseases such as Pneumococcal, Meningococcal or Haemophilus influenzae diseases.

The production of purified N. meningitidis capsular polysaccharides is the foremost requirement for an effective conjugation with the carrier protein and its development as a conjugate vaccine. Most of the bacterial fermentation media traditionally use animal components for growth of meningococcal bacteria for polysaccharide production. An animal component free medium will be desired to provide advantage in terms of regional preferences and to avoid infectious agents causing diseases e.g. Transmissible spongiform encephalopathy (TSE) and bovine spongiform encephalopathy (BSE). The cost for the cultivation of N. meningitidis for production of capsular polysaccharides is generally high and involves long working hours since it involves a series of production and quality control steps. An optimized animal component free medium can obviate these issues.

Improvement in the polysaccharide production steps would lead to formulation of efficacious and economically viable conjugate vaccines.

There are a number of patents and non-patent disclosures that describe the processes of production and purification of polysaccharides. One such disclosure is patent application no. U.S. Ser. No. 12/041,745 discloses a method of producing a meningococcal meningitis vaccine, the method, includes culturing N. meningitidis to produce capsular polysaccharides of serogroups A, C, Y and W-135 in N. meningitidis fastidious medium (NMFM), isolating the capsular polysaccharides from the culture, purifying the capsular polysaccharides of any residual cellular biomass; and depolymerizing the capsular polysaccharide mechanically. The cited art utilizes long hours for the production of purified capsular polysaccharide.

Another US patent publication no. US 20150299750 A1 discloses an improved culture, fermentation and purification conditions for preparing Neisseria meningitidis polysaccharides. Another US patent publication no.: 20080318285 A1 discloses Neisseria meningitidis fastidious medium designed to maximize the yield of capsular polysaccharides and generate minimal cellular bio mass and endotoxin in a short duration of fermentation.

ACFM (Animal component free media) of the present invention is different from that used by Shankar Pisal in US patent publication no. US 2015/0299750 and Jeeri reddy in US patent publication no.: US 2008/0318285, none of above prior arts have used Select phytone and TC yeastolate. Select Phytone™ is a peptone of plant origin. The nitrogen content of the phytone combined with the naturally occurring vitamins supports bacterial growth. Phytone has an endotoxin level of less than or equal to 500 EU/g. The TC Yeastolate is a mixture of peptides, amino acids, carbohydratesas well as vitamins. The TC Yeastolate products are animal component-free and water-soluble portions of autolyzed yeast. TC Yeastolate, UF has been ultrafiltered at a 10,000 MWCO (Molecular Weight Cut-Off). It has an endotoxin value of less than 500 EU/g. It is a versatile nutritional supplement which enhances bacterial growth promotion. It is a new class of media component which replaces most of the individual components used for growth promotion. The present media composition of the invention does not include even casamino acid which has been used in prior art by other inventors. The biggest advantage of animal component free media is that ACFM vaccines (millions of doses) are in high demand in middle east, GCC (Gulf cooperation council) countries and other countries because due to regional preferences, further such vaccines are free from BSE and TSE.

Therefore, the various methods used for the production of N. meningitidis serogroups presently utilize animal component media and take relatively long time of upto 20-24 hours or more for the fermentataion process for the cultivation of polysaccharide thereby increasing the cost of production and making the process commercially less feasible since they cannot be scaled up in a cost-effective and timely manner and have animal components.

It is an object of the present invention to provide improved culture media and feed media, for better production of N. meningitidis polysaccharides by fermentation in reduced time and with high yields. Said improvements will result in manufacturing polysaccharide protein conjugate vaccine at lesser price and subsequently vaccine can be made available to children of developing countries at an affordable rate.

### OBJECT OF THE INVENTION

The main object of the present invention is to provide a process of production of bacterial polysaccharide.

Another object of the present invention is to provide a process of production of capsular polysaccharides of various serogroups of Neisseria meningitidis.

Yet another object of the present invention is to provide optimized culture media and feed media composition which is free from animal component.

Yet another object of the present invention is to provide improved culture media and feed media composition for growth of fastidious Neisseria meningitidis serogroups A, C, W, X and Y.

Yet another object of the present invention is to provide process of fermentation in reduced time with better polysaccharide yield with low impurities in a very short time by simple, efficient, improved and commercially scalable methods.

Yet another object of the present invention is to purify Neisseria meningitidis polysaccharides, while eliminating impurities in a very short time by simple, efficient, improved and commercially scalable methods.

Yet another object of the present invention is to produce high quality product with better yield that meet the relevant quality specifications.

### SUMMARY OF THE INVENTION

The present invention describes a rapid, industrially scalable, cost effective process for growth of bacteria preferably Neisseria meningitidis for production of bacterial polysaccharide. The said process provides a purification method for purifying N. meningitidis polysaccharide at a significantly reduced time.

The present invention describes culture media for N. meningitidis including but not limited to monosodium glutamate in a concentration range of 1.00±0.5 g/L, di-sodium hydrogen phosphate in the range of 3.25±1.0 g/L, potassium chloride in the range of 0.09±0.1 g/L, select phytone in the range of 10.0±2.0 g/L, yeastolate in the range of 4.0±2.0 g/L, dextrose in the range of 5.00±2.0g/L, L-cystine in the range of 0.03±0.1 g/L, magnesium chloride in the range of 0.60±0.5 g/L, nicotinamide adenine dinucleotide in the range of 0.25±0.1 g/L and ammonium chloride in the range of 1.00±0.2 g/L composition. The above-mentioned culture media composition provides optimal growth for N. meningitidis serogroups.

The present invention also describes feed media for N. meningitidis including but not limited to L-glutamic acid in the range of 6.00±2.0g/L, dextrose in the range of 20±2.0 g/L, L-serine in the range of 0.50±0.1 g/L, L-arginine in the range of 0.20±0.1 g/L, glycine in the range of 0.20±0.1 g/L, L-tryptophan in the range of 0.20±0.1 g/L, TC-yeastolate in the range of 5±2.0 g/L and other components like L-cystine, magnesium chloride, calcium chloride, ferrous sulphate, ammonium chloride as per the requirement. The above-mentioned feed media composition provides optimal growth for N. meningitidis serogroups when added in the fermentation broth during fermenter culture with the aforementioned culture media.

The present invention describes the fermentation process at predetermined temperature, pH, airflow, dissolved oxygen and rate of agitation, such that the fermentation is completed within 11±3 hours.

The present invention describes purification steps for producing high yields of N. meningitidis serogroup W and Y capsular polysaccharides. The crude polysaccharide from the fermentation broth is subjected to concentration and diafiltration against MilliQ water (MQW) to form a concentrate with reduced impurity level. The concentrate so obtained is subjected to treatment with an alkali such as NaOH at 1±0.2 M at predetermined temperature for an optimized time. The resultant partially purified polysaccharide is subjected again to diafiltration with MQW followed by carbon filtration and finally subjected to sterile filtration.

### BRIEF DESCRIPTION OF DRAWINGS

FIG. 1 depicts growth curves for Shake Flask Study 1 (6 ACFM compositions).

FIG. 2 depicts growth curves for Shake Flask Study 2 (5 ACFM and 1 ACM compositions).

FIG. 3 depicts growth curves of MenA batches with ACFM.

FIG. 4 depicts growth curves of MenC batches with ACFM.

FIG. 5 depicts growth curves of MenY batches with ACFM.

FIG. 6 depicts growth curves of MenW batches with ACFM.

FIG. 7 depicts growth curves of MenX batches with ACFM.

### DETAILED DESCRIPTION OF THE INVENTION

The invention discloses optimized culture media and feed media which is free from animal component for growth of fastidious Neisseria meningitidis in lesser time.

The biggest advantage of animal component free media is that ACFM vaccines (millions of doses) are in high demand in the Middle East, the GCC (Gulf cooperation council) countries and other countries due to regional preferences of ACFM vaccines. Further, such vaccines are free from TSE and BSE risks.

Before the preferred embodiment of the present invention is described, it is understood that this invention is not limited to the particular materials described, as they may vary. It is also understood that the terminology used herein is for the purpose of describing the particular embodiment only and is not intended to limit the scope of the invention in any way.

The present invention describes culture media for N. meningitidis including but not limited to monosodium glutamate in a concentration range of 1.00±0.5 g/L, di-sodium hydrogen phosphate in the range of 3.25±1.0 g/L, potassium chloride in the range of 0.09±0.1 g/L, select phytone in the range of 10.00±2.0 g/L, yeastolate in the range of 4.00±2.0 g/L, dextrose in the range of 5.00±2.0g/L, L-cystine in the range of 0.03±0.1 g/L, magnesium chloride in the range of 0.60±0.5 g/L, nicotinamide adenine dinucleotide in the range of 0.25±0.1 g/L and ammonium chloride in the range of 1.00±0.2 g/L composition. The above-mentioned culture media composition provides optimal growth for N. meningitidis serogroups.

The above-mentioned culture media composition provides optimal growth for N. meningitidis serogroups.

In a preferred embodiment the present invention describes culture media for N. meningitidis comprising monosodium glutamate in a concentration of 1.00 g/L, di-sodium hydrogen phosphate in the concentration of 3.25 g/L, potassium chloride in the concentration of 0.09 g/L, select phytone in the concentration of 10.00 g/L, TC-yeastolate in the concentration of 4 g/L, dextrose in the concentration of 5.00 g/L, L-cystine in the concentration of 0.03 g/L, magnesium chloride in the concentration of 0.60 g/L, nicotinamide adenine dinucleotide in the concentration of 0.25 g/L and ammonium chloride in in the concentration of 1.00 g/L.

All the above optimized concentrations are listed in Table 4 of the specification. The above-mentioned culture media composition provides optimal growth for N. meningitidis serogroups MenA, MenC, MenY, MenW and MenX. Ammonium chloride is added only to Serogroups W and X ACFM for optimal polysaccharide production.

The present invention also describes feed media for N. meningitidis including but not limited to L-glutamic acid in the range of 6.00±2.0g/L, dextrose in the range of 20.00±2.0 g/L, L-serine in the range of 0.50±0.1 g/L, L-arginine in the range of 0.20±0.1 g/L, glycine in the range of 0.20±0.10 g/L, L-tryptophan in the range of 0.20±0.1 g/L, TC-yeastolate in the range of 5.00±2.0 g/L. The above-mentioned feed media composition provides optimal growth for N. meningitidis serogroups.

In a preferred embodiment the present invention also describes feed media for N. meningitidis comprising L-glutamic acid in the concentration of 6.00 g/L, dextrose in the concentration of 20.00 g/L, L-serine in the concentration of 0.50 g/L, L-arginine in the concentration of 0.20 g/L, glycine in the concentration of 0.20 g/L, L-tryptophan in the concentration of 0.20 g/L, TC-yeastolate in the concentration of 5.00 g/L. The above-mentioned feed media composition provides optimal growth for N. meningitidis serogroups MenA, MenC, MenY, MenW and MenX. The optimized feed composition is listed in Table 6 of the specification.

After growth of bacteria in flask with optimized culture media, the bacteria are subjected to fermentation as disclosed in Example 5 and Example 6 of the specification. The fermentation conditions are so optimized that the resultant fermentation harvest (broth) have high polysaccharide yield and low level of impurities and the fermentation process is completed within 11±3 hours, more preferably 10 to 12 hours.

In a preferred embodiment, the fermentation is carried out in a temperature range of 36±1° C. with rpm in the range of 150 to 600 rpm, the air flow of the fermenter is maintained at 0.2 to 0.8 l/m and the partial pressure of Oxygen (PO₂) is maintained at 20% for throughout the fermentation along with a pH of 7.2±0.1.

Therefore, the present invention provides a rapid, industrially scalable, cost effective process for the production of Neisseria meningitidis serogroups MenA, MenC, MenY, MenW and MenX with optimized culture media and feed media which provides maximum growth to the Neisseria meningitidis.

Various aspects of the invention described in detailed above is now illustrated with non-limiting examples:

### Example-1: Shake Flask Experiments for the ACFM Optimization

### Shake Flask Study 1:

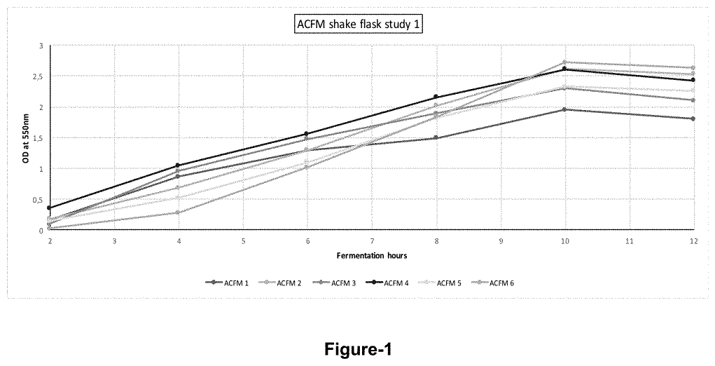

Six shake flasks each having different compositions of animal component free media (ACFM) as diclsoed in Table-1 are used for media optimization of Neisseria meningitidis serogroup W (MenW). The OD_{550 nm} of flask culture is recorded after every 2 hours, until 12^{th} hour for all the six flasks. The growth curves are presented in FIG. 1. The culture samples are inactivated at the 10^{th} hour with 1% v/v formalin and are tested for the polysaccharide (PS) concentration using inhibition ELISA for all the six flasks. As there is a decline in the OD_{550 nm} in all the six shake flasks after 10^{th} hour, therefore the samples at the 10^{th} hour (late log phase of bacterial growth) is selected for the estimation of PS concentration by inhibition ELISA as described below in Example 2. It is observed that ACFM 1 and ACFM 3 gave high PS concentration. Moreover, ACFM 3 gives high PS concentration with high OD_{550 nm} as compared to ACFM 1. The PS concentration of the all the six ACFM is described in Table-2. Based on the combination of high PS concentration and high OD_{550 nm}, in addition to the requirement of lesser media components, ACFM 3 is short listed as lead medium from MenW shake flask experiments.

TABLE 1
Shake flask Study 1 (ACFM compositions)

| | | ACFM 1 | ACFM 2 | ACFM 3 | ACFM 4 | ACFM 5 | ACFM 6 |
|---|---|---|---|---|---|---|---|
| S. no. | Media Composition | g/L | g/L | g/L | g/L | g/L | g/L |
| 1 | Sodium phosphate | 3.25 | 3.25 | 3.25 | 3.25 | 3.25 | 3.25 |
| | dibasic | | | | | | |
| 2 | Sodium dihydrogen | 1.625 | 1.625 | NA | 1.625 | NA | NA |
| | phosphate dihydrate | | | | | | |
| 3 | KCl | 0.09 | 0.09 | 0.09 | 0.09 | 0.09 | 0.09 |
| 4 | Select Phytone | 10 | 15 | 10 | 10 | 15 | 20 |
| 5 | TC Yeastolate | 10 | 4 | 4 | 4 | 15 | 4 |
| 6 | Mono sodium | 1 | 1 | 1 | 1 | 1 | 1 |
| | glutamate | | | | | | |
| 7 | Dextrose | 15 | 10 | 5 | 5 | 20 | 5 |
| 8 | L-Cystine | 0.03 | 0.03 | 0.03 | 0.03 | 0.03 | 0.03 |
| 9 | Magnesium sulfate | 0.6 | 0.6 | 0.6 | 0.6 | 0.6 | 0.6 |
| 10 | Ammonium chloride | 1 | 1 | 1 | 1 | 1 | 1 |
| 11 | NaCl | NA | 1 | NA | 2 | NA | NA |
| 12 | NAD | 0.25 | 0.25 | 0.25 | 0.25 | 0.25 | 0.25 |
| 13 | β- alanine | NA | 0.1 | NA | NA | 0.1 | 0.1 |
| 14 | Thiamine HCl | 0.1 | NA | NA | NA | 0.1 | 0.1 |
| 15 | Vit. B 12 | NA | NA | NA | 0.1 | NA | 0.1 |

NA—Not applicable

TABLE 2
PS concentration in the shake flasks

| | PS | | | | | |
|---|---|---|---|---|---|---|
| Fermentation | concentration | PS conc | PS conc | PS conc | PS conc | PS conc |
| hours | (conc) ACFM 1 | ACFM 2 | ACFM 3 | ACFM 4 | ACFM 5 | ACFM 6 |
| 10 | 13.9 μg/ml | 7.4 μg/ml | 10.7 μg/ml | 6.6 μg/ml | 5.3 μg/ml | 7.4 μg/ml |

### Shake Flask Study 2:

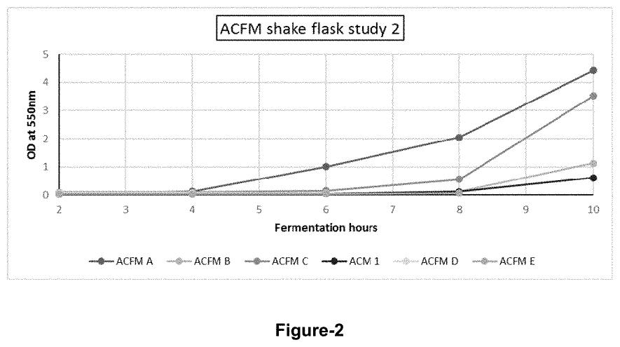

Flask Study 2 is conducted for MenC ACFM optimization with six different media compositions (Table-3) out of which five compositions had ACFM and one had animal component containing medium (ACM). The OD_{550 nm} is recorded after every 2 hours, until 10^{th} hour for all the six flasks. The growth curves are described in FIG. 2 and the media compositions in Table-3. ACFM A (similar to ACFM 3 of shake flask study 1) gave the highest OD_{550 nm} compared to other media compositions of shake flasks study 2.

Based on the Shake Flask Study 1 and Study 2, ACFM 3 of Study 1 which is similar to ACFM A of study 2 is selected for scale-up/fermentation experiments for all serogroup A, C, Y, W and X. The said media composition is relatively simpler and cost effective as compared to other ACFM supporting good growth and PS production.

TABLE 3
Shake flask Study 2 (5 ACFM and 1 ACM compositions)

| | | ACFM A | ACFM B | ACFM C | ACM 1 | ACFM D | ACFM E |
|---|---|---|---|---|---|---|---|
| S. no. | Media Composition | g/L | g/L | g/L | g/L | g/L | g/L |
| 1 | Sodium phosphate | 3.25 | 3.25 | 3.25 | 3.25 | 3.25 | 3.25 |
| | dibasic | | | | | | |
| 2 | Sodium dihydrogen | NA | 1.625 | 1.625 | 1.625 | 1.625 | 1.625 |
| | phosphate dihydrate | | | | | | |
| 3 | KCl | 0.09 | NA | 0.09 | 0.09 | NA | NA |
| 4 | Select Phytone | 10 | 10 | 10 | 10 | 10 | 10 |
| 5 | TC Yeastolate | 4 | 4 | NA | NA | NA | 4 |
| 6 | Mono sodium glutamate | 1 | 1 | 1 | 1 | NA | 1 |
| 7 | Dextrose | 5 | 5 | 8 | 8 | 5 | 5 |
| 8 | L-Cystine | 0.03 | 0.03 | 0.03 | 0.03 | NA | 0.03 |
| 9 | Magnesium sulfate | 0.6 | NA | NA | NA | NA | 0.6 |
| 10 | Glycine | NA | 0.2 | NA | NA | NA | NA |
| 11 | NAD | 0.25 | 0.25 | 0.25 | 0.25 | NA | 0.25 |
| 12 | L-Arginine | NA | 0.2 | NA | NA | NA | NA |
| 13 | Thiamine HCl | NA | 0.2 | NA | NA | NA | NA |
| 14 | Casamino acid | NA | NA | NA | 10 | NA | NA |
| 15 | Yeast extract | NA | NA | 10 | NA | 10 | NA |
| 16 | NaCl | NA | NA | NA | NA | NA | 1 |

### Example-2: Inhibition ELISA Protocol

Inhibition ELISA method was used for estimation of the polysaccharide content in the bacterial culture broth. In this the sample containing meningococcal capsular polysaccharide is incubated with the serogroup specific polyclonal antibody (primary antibody) so that complexes will be formed between the antibody and antigens in the sample. These complexes are then added to a container in which competitor homologous antigens are immobilized. Antibody which is not complexed with immunogens from the polysaccharide test sample bind to these immobilized competitor antigens. The antibody which is bound to the immobilized competitor antigens (after usual washing steps, etc.) can then be detected by adding an enzyme labelled secondary antibody which binds to the primary antibody. The label is used to identify the reaction of immobilized primary antibody to secondary antibody utilizing a chromogenic substrate. The reduction in the absorbance in test well as compared to the control well (without any test sample) confirms the presence of the specific antigen in the test sample and the percentage inhibition of the antibody is directly proportional to the polysaccharide concentration in the test sample.

Briefly, the ELISA is performed, wherein the Plate A is coated with 100 μl of coating solution having equal volume of in-house PS and mHSA and incubated for overnight at 2-8° C. A no-antigen-control is included as control. The coated plate is blocked at room temperature with 200 μl of blocking buffer. Quality control polysaccharide (Standard) of defined concentration are serially diluted three-fold as are the bacterial culture supernatant (test samples) and incubated in Plate B with serogroup specific polyclonal primary antibody for 1 hour at 37° C. The antigen-antibody mixture from Plate B is transferred to blocked Plate A and further incubated for two hours (1.5 hours at 37° C. and half an hour at room temperature). The plate is further incubated with secondary antibody for 1 hour and reaction is developed using 100 μl of TMB substrate and incubated for 10 min. The reaction is stopped with 50 μl of 2M H₂SO₄ per well before OD at 450 nm is observed with reference to 630 nm. The inhibition percentage is calculated from inhibition of OD in standard or test sample dilutions in relation to OD of no-antigen control wells. The standard curve is generated from inhibition percentages for quality control dilutions which is used to extrapolate the concentration of polysaccharide in the test samples using Combistat software.

### Example-3: Optimized ACFM Composition

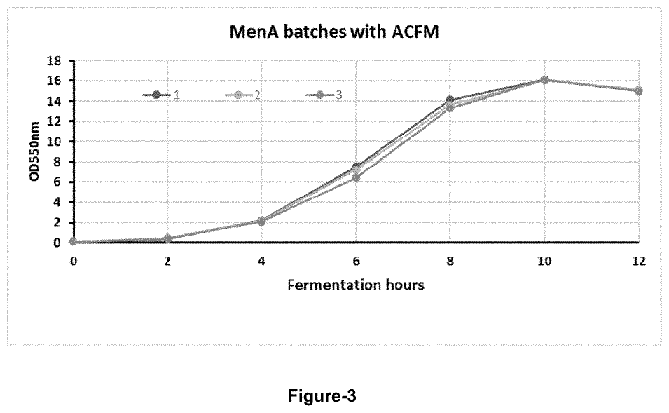

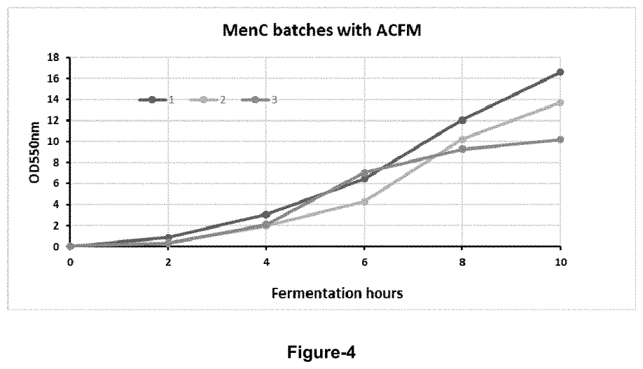

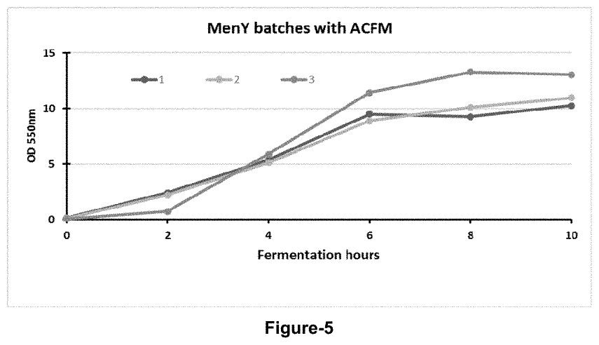

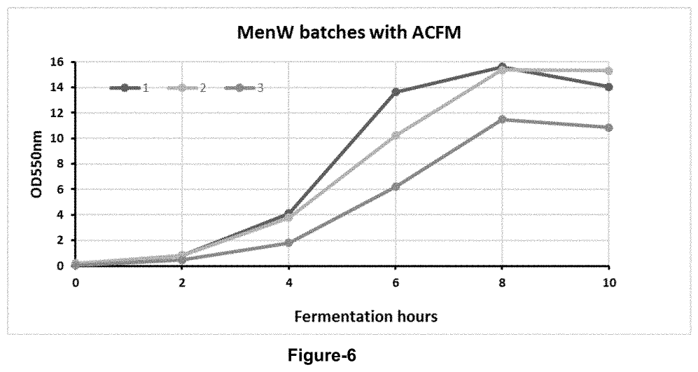

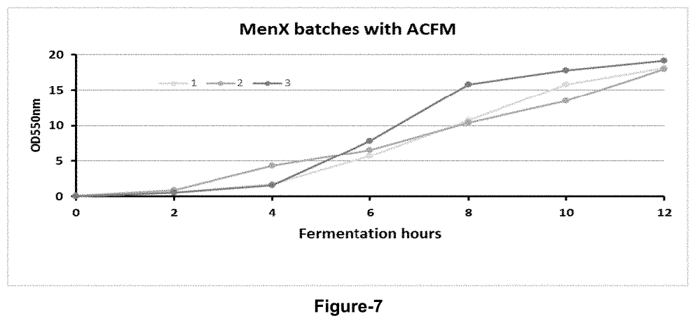

ACFM composition selected on the basis of Study 1 and 2 shake flask experiments is taken forward for scale-up/fermentation experiments (2.5 L scale) each for Men A, C, Y, W and X serogroups. All the serogroups gave rise to good growth (FIG. 3-7). The final ACFM culture media composition is described in Table-4 below. Serogroups W and X ACFM gave optimal growth with addition of ammonium chloride.

TABLE 4
Final ACFM Culture Media composition

| | | Concentration |
|---|---|---|
| S. no. | Component | (g/L) |
| 1 | Na₂HPO₄ anhydrous | 3.25 |
| | (di-sodium hydrogen phosphate) | |
| 2 | KCl (potassium chloride) | 0.09 |
| 3 | Select phytone | 10.0 |
| 4 | TC- Yeastolate | 4.0 |
| 5 | Dextrose | 5.00 |
| 6 | NH₄Cl (ammonium chloride) (for MenW | 1.00 |
| | and MenX | |
| 7 | C₅H₈NO₄Na (mono sodium glutamate) | 1.00 |
| 8 | MgSO₄ (magnesium chloride) | 0.60 |
| 9 | L- Cystine | 0.03 |
| 10 | NAD (Nicotinamide adenine dinucleotide) | 0.25 |

### Example-4: Feed Composition

The Feed composition is designed after performing fermentation experiments using ACM 2 as disclosed in Table-5 for the production of MenX (out of several other media optimization experiments for MenX which did not provide optimal growth and/or polysaccharide yields). The final ACFM feed components are defined in Table-6. The ACFM feed components are referred from and designed using the feed components of MenX ACM 2 experiment and ACFM fermentation medium. The ACFM feed components are cost effective and are required for the optimal bacterial growth.

TABLE 5
ACM 2 composition for MenX fermentation experiments
ACM 2

| S. no. | Media Composition | g/L |
|---|---|---|
| 1 | Dextrose | 10 |
| 2 | Sodium chloride | 5.8 |
| 3 | Potassium sulfate | 1 |
| 4 | Potassium phosphate di basic | 4 |
| 5 | Ammonium chloride | 0.15 |
| 6 | Glutamic acid | 5 |
| 7 | Arginine | 0.3 |
| 8 | Serine | 0.5 |
| 9 | Cysteine | 0.25 |
| 10 | Magnesium chloride | 0.19 |
| 11 | Calcium chloride | 0.02 |
| 12 | Ferrous sulphate | 0.002 |
| 13 | Casamino acid | 10 |
| | Feed | g/L |
| 1 | Dextrose | 75 |
| 2 | L-Glutamic acid | 40 |
| 3 | L-Arginine | 3 |
| 4 | L-Serine | 3 |
| 5 | Cysteine | 2 |
| 6 | Magnesium chloride | 2 |
| 7 | Calcium chloride | 0.15 |
| 8 | Ferrous sulphate | 0.02 |

TABLE 6
Finalized ACFM Feed Media composition

| S. no. | Components | Concentration (g/L) |
|---|---|---|
| 1 | L-glutamic acid | 6.00 |
| 2 | Dextrose | 20.00 |
| 3 | L-serine | 0.50 |
| 4 | L-Arginine | 0.20 |
| 5 | Glycine | 0.20 |
| 6 | L-tryptophan | 0.20 |
| 7 | TC- Yeastolate | 5.00 |

The above-mentioned ACFM feed composition as listed in Table-6 is unique and different from that used by inventors in prior art (Shankar Pisal, US 2015/0299750 and Jeeri Reddy, US 2008/0318285) and supports better growth of all serogroups (MenA, MenC, MenY, MenW and MenX).

The nutrient fermentation media and feed components utilized in the present invention lead to low cellular biomass production with low levels of endotoxins and thus result in polysaccharide which has minimal level of impurities in the harvested fermentation broth.

### Example 5: Fermentation Procedure

One vial from working cell bank is thawed and is streaked onto ACFM agar plates. The plates are incubated for overnight at 36±1° C. and at 5±1% CO₂. The media composition for the preparation of ACFM plates is same as described in Table-4 with an addition of agar at a concentration of 15 g/L.

After overnight incubation of the plate, ACFM flask is inoculated with culture from ACFM agar plate and incubated at 36±1° C., 5±1% CO₂, and 150 rpm until growth appears. When the growth reaches an OD_{550 nm} of 1±0.1, the flask culture is aseptically inoculated into the fermenter and the fermentation started with preset conditions as mentioned in Example 6.

When OD of culture in the fermenter reaches 1±0.1 (generally after the initial 2.5-3 hours of fermentation), feed is added at the rate of 1±0.2 ml/min. Total 500-600 ml feed for 2.5L fermentation media is prepared and is utilized within the next 8-10 hours of fermentation. The total fermentation time is generally in the range of 11±3 hours. OD_{550 nm} is monitored every two hours to observe the growth.

### Example 6: Fermentation Conditions

The fermentation is carried out in optimized conditions as enumerated in Table 7 below:

TABLE 7
Fermenter conditions

| Parameters | Range |
|---|---|
| Temperature | 36 ± 1° C. |
| Rpm | 150 rpm to 600 rpm |
| Air flow | 0.2 to 0.8 l/m |
| pH | 7.2 ± 0.1 |
| PO₂ | Actual level in starting and maintaining |
| | at 20% throughout fermentation hours |

### Example 7: Inactivation and Harvesting Fermentation Broth

The indicator to inactivate the fermentation is either the decline in OD_{550 nm} after reaching peak or increase in pH or both. Inactivation is carried out using 1±0.2% v/v formalin for 4±1 hours at 36±1° C., and the harvesting is done by centrifugation at 10550×g for 30 minutes. The supernatant is collected and stored at 2-8° C. and utilized within 24 hours preferably immediately upon harvest for the purification of PS.

The animal component containing medium (ACM) is compared with ACFM for the MenA serogroup in fermenter culture. The animal component free media (ACFM) gave higher yields when MenA fermentation is conducted using ACFM composition compared to ACM composition. The average purified PS yield for MenA using ACFM is 699 mg/L, whereas it is 330 mg/L fermentation broth using ACM. The OD_{550 nm} for the ACFM for various serogroups, is higher in general when compared to that with the ACM.

The Polysaccharide concentration in the fermentation broth at different time intervals for all the serogrouos were anlaysed, the results for MenX by Inhibition ELISA is shown below in Table-8.

TABLE 8
MenX PS concentration during fermentation

PS Conc. (mg/L)

| | | | |
|---|---|---|---|
| Sample | Batch 1 | Batch 2 | Batch 3 |

| | | | | |
|---|---|---|---|---|
| 2 | Hrs | 18 | 18 | 31 |
| 4 | Hrs | 56 | 48 | 42 |
| 6 | Hrs | 198 | 184 | 125 |
| 8 | Hrs | 519 | 347 | 254 |
| 10 | Hrs | 587 | 763 | 468 |
| 10.5-11 | Hrs | 930 | | 556 |

| | | | |
|---|---|---|---|
| Fermentation broth at Harvest | 1012 | 1288 | 718 |

The yield as obtained by inhibition ELISA at the time of harvest after the fermentataion process is shown below in Table-9.

TABLE 9
PS yields by inhibition ELISA at time of harvest after fermentation

| Serogroup | Yield |
|---|---|
| MenA | upto 2050 mg/L fermentation broth |
| MenC | upto 600 mg/L fermentation broth |
| MenY | up to 1850 mg/L fermentation broth |
| MenW | up to 400 mg/L fermentation broth |
| MenX | up to 1288 mg/L fermentation broth |

Thus, the present invention provides improved culture and feed media, for better production of N. meningitidis polysaccharides by fermentation in reduced time with high yields.

We claim:

1. A process for the production of Neisseria meningitidis polysaccharides of serogroups A, C, Y, W and X with improved animal component free culture media, improved animal component free feed media and improved fermentation process parameters for rapid growth of the Neisseria meningitides with enhanced yield.

2. The process as claimed in claim 1 wherein said improved animal component free culture media for the production of Neisseria meningitidis polysaccharides of serogroups A, C and X comprises of combination of two or more ingredients selected from following:
Monosodium glutamate
Di-sodium hydrogen phosphate
Potassium chloride
Select phytone
TC-yeastolate
Dextrose
L-cystine
Magnesium chloride
Nicotinamide adenine dinucleotide
wherein said combination provides for rapid growth of Neisseria meningitidis polysaccharides of serogroups A, C and X with enhanced yield.

3. The process as claimed in claim 2 wherein said culture media comprises of the following in the concentration range of:

| Ingredients | Concentration (g/L) |
|---|---|
| Monosodium glutamate | 1.00 ± 0.5 |
| Di-sodium hydrogen phosphate | 3.25 ± 1 |
| Potassium chloride | 0.09 ± 0.1 |
| Select phytone | 10.0 ± 2 |
| TC-yeastolate | 4.0 ± 2 |
| Dextrose | 5.00 ± 2 |
| L-cystine | 0.03 ± 0.1 |
| Magnesium chloride | 0.60 ± 0.5 |
| Nicotinamide adenine dinucleotide | 0.25 ± 0.1. |

4. The process as claimed in claim 1 wherein said improved animal component free culture media for the production of Neisseria meningitidis polysaccharides of serogroups Y and W comprises of combination of two or more of:
Monosodium glutamate
Di-sodium hydrogen phosphate
Potassium chloride
Select phytone
TC-yeastolate
Dextrose
L-cystine
Magnesium chloride
Nicotinamide adenine dinucleotide
Ammonium chloride
wherein said combination provides for rapid growth of Neisseria meningitidis polysaccharides of serogroups Y and W with enhanced yield.

5. The process as claimed in claim 4 wherein said culture media comprises of the following in the concentration range of:

| Ingredients | Concentration (g/L) |
|---|---|
| Monosodium glutamate | 1.00 ± 0.5 |
| Di-sodium hydrogen phosphate | 3.25 ± 1 |
| Potassium chloride | 0.09 ± 0.1 |
| Select phytone | 10.0 ± 2 |
| TC-yeastolate | 4.0 ± 2 |
| Dextrose | 5.00 ± 2 |
| L-cystine | 0.03 ± 0.1 |
| Magnesium chloride | 0.60 ± 0.5 |
| Nicotinamide adenine dinucleotide | 0.25 ± 0.1 |
| Ammonium chloride | 1.00 ± 0.2. |

6. The process as claimed in claim 1 wherein said improved feed media for the production of Neisseria meningitidis polysaccharides of serogroups A, C, Y, W and X comprises of combination of two or more of:
L-glutamic acid
Dextrose
L-serine
L-arginine
Glycine
L-tryptophan
TC-yeastolate
wherein said combination provides for rapid growth of Neisseria meningitidis polysaccharides with enhanced yield.

7. The process as claimed in claim 6 wherein said feed media comprises of the following in the concentration range of:

| Ingredients (fermentation broth) | Concentration (g/L) |
|---|---|
| L-glutamic | 6.00 ± 2 |
| Dextrose | 20.00 ± 2 |
| L-serine | 0.50 ± 0.1 |
| L-arginine | 0.20 ± 0.1 |
| Glycine | 0.20 ± 0.1 |
| L-tryptophan | 0.20 ± 0.1 |
| TC-yeastolate | 5.00 ± 2. |

8. The process as claimed in claim 1 wherein said culture media and feed media provides enhanced yields of Neisseria meningitidis polysaccharides of serogroup A, C, Y, W and X in the following concentrations:

| Serogroup | Yield |
|---|---|
| MenA | upto 2050 mg/L fermentation broth |
| MenC | upto 600 mg/L fermentation broth |
| MenY | up to 1850 mg/L fermentation broth |
| MenW | up to 400 mg/L fermentation broth |
| MenX | up to 1288 mg/L fermentation broth |

9. The process as claimed in claim 1 wherein said fermentation process is carried out in the following process parameters range:

| Parameters | Range |
|---|---|
| Temperature | 36 ± 1° C. |
| Rpm | 150 rpm to 600 rpm |
| Air flow | 0.2 to 0.8 l/m |
| pH | 7.2 ± 0.1 |
| PO₂ | Actual level in starting and maintaining |
| | at 20% throughout fermentation hours |

10. The process of ferementation as claimed in claim 9 wherein said fermentation process is achieved rapidly in 11+3 hours.

11. The process for the production of Neisseria meningitidis polysaccharides as claimed in claim 1, wherein said process results in N. meningitidis polysaccharides which are capable of being used in the production of economical polysaccharide protein conjugate vaccine(s) against meningococcal infections.
